(19) United States
(12) Patent Application Publication
(10) Pub. No.: US 2007/0099993 A1
(43) Pub. Date: May 3, 2007

(54) Antimicrobial and anticancer properties of methyl-beta-orcinolcarboxylate from lichen (Everniastrum cirrhatum)

(75) Inventors: Suman Preet Singh Khanuja, Lucknow (IN); Ranganathan Santha Kumar Tiruppadiripuliyur, Lucknow (IN); Vivek Kumar Gupta, Lucknow (IN); Preeti Chand, Lucknow (IN); Ankur Garg, Lucknow (IN); Santosh Kumar Srivastava, Lucknow (IN); Subash Chandra Verma, Lucknow (IN); Dharmendra Saikia, Lucknow (IN); Mahendra Pandurang Darokar, Lucknow (IN); Ajit Kumar Shasany, Lucknow (IN); Anirban Pal, Lucknow (IN)

Correspondence Address: LADAS & PARRY, 26 WEST 61ST STREET, NEW YORK, NY, 10023, US

(73) Assignee: COUNCIL OF SCIENTIFIC AND INDUSTRIAL RESEARCH

(21) Appl. No.: 11/635,339

(22) Filed: Dec. 7, 2006

Related U.S. Application Data
(62) Division of application No. 10/404,012, filed on Mar. 31, 2003.

(51) Int. Cl. A61K 31/235

(52) U.S. Cl. 514/543

(57) ABSTRACT

The present invention relates to the new use of an already known biomolecule methyl-β-orcinol carboxylate of formula I isolated from a lichen (Everniastrum cirrhatum), for treating pathogenic fungal infections of humans that are resistant to polyene and azole antibiotics such as amphotericin B, nystatin, clotrimazole etc.

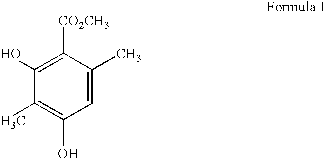

### FIELD OF THE INVENTION

The present invention relates to the new use of an already known biomolecule methyl-β-orcinol carboxylate of formula I isolated from a lichen (Everniastrum cirrhatum), for treating pathogenic fungal infection of humans that are resist to polyene and azole antibiotics-such as amphotericin B, nystatin, clotrimazole etc.

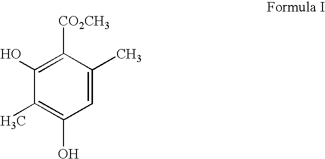

### BACKGROUND OF THE INVENTION

Lichens are symbiotic associations between fungi, green algae and/or cyanobacteria They have a varied chemistry and produce many polyketide-derived compounds, including some, such as depsides and depsidones that are rarely reported elsewhere. Depsides are a class of compounds, which appear to be unique to the lichens. These compounds are dimeric esters of variously substituted orsellinic acids and are the major source of the so-called lichen acids. Although lichens have been appreciated in traditional medicines, their value has largely been ignored by the modem pharmaceutical industry because difficulties in establishing axenic cultures and conditions for rapid growth preclude their routine use in most conventional screening processes.

The association between fungi and algae is specific and selective. The name of the fill component is given to the whole lichen and there are >13500 described species, including almost one fifth of all known fungi (Hawcksworth and Hill, 1984; The Lichen Forming Fungi, Mecorquodale Ltd). Although the individual mycobionts and photobionts (the fungi and the photosynthetic algae or cyanobacteria, respectively) are small and nondescript if culture in a laboratory dish, the symbiotic components together in nature present a full rage of varied and beautiful forms, and some such as Ramalina menziesii (the ‘fishnet’ lichen) can drape entire trees, creating a prominent display (Arvis, W. O., 2000; Lichens, Smithsonian Situation Press). They perform a variety of ecological roles such as colonizing margin habitat in Antarctica, stability soil in the semi-arid desert of Australia and contributing to nitrogen turnover in the northern pacific forests of North America.

They produce characteristic secondary metabolites that are unique with respect to those of high plants. Lichens produce a wide range of chemical compounds, among which appropriately 350 secondary metabolites have been identified. These mycobionts derived products usually accumulate as extra cellular crystals on the cell walls of the symbiosis, and account for up to 10% (in exceptional cases, up to 40%) of thallus dry mass (Gahin, M. and Shomer-llan, A (1988) in CRC Handbook of Lichenology, Vol. III; Galun, M, ed.), pp, 3-8. CRC Press); many are unique to lichens. Most lichen secondary compounds are formed by the polyketide pathway, while others derive from the shikimic acid and movalonic acid pathways these are key routes for secondary metabolism in all organisms. Several lichen extracts have been used for various remedies in folk medicine, and screen test with lichens have indicated the frequent occur of metabolites with antibiotic, antimycobacterial, antiviral, analgesic, and antipyretic properties.

Furthermore, a distinct class of lichen metabolites is the depsides. These types of compounds are formed by condensation of two or more hydroxybenzoic acids whereby the carboxyl group of one molecule is esterified with a phenolichydroxyl group of a second molecule. Owing to the phenolic nature of their chemical structures, these molecules are interesting candidates for evaluating their effects on leukotriene biosynthesis, as a major class of inhibitors often contains a hydroxylated armatic ring (Fitzsimmons et al 1989). Moreover, two small-molecule lichen-derived metabolites, protolichesterinic acid and lobaric acid, have been reported to inhibit 5-LO from porcine leukocytes (Ogmundsdottir et al 1998). The latter has also been shown to inhibit peptide leukotriene formation (Gissurarson et al 1997). Lichen depsides have also been described to inhibit prostaglandin biosynthesis (Sankawa et al 1982).

Lichens and lichen products have been used in traditional medicines for Vies and still hold considerable invest as alternative treatments in various parts of the world. Indeed, today a variety of lichen-based tonics, lotions and lozenges call be purchased in Iceland, where they're medicinal. However, lichens have been essentially ignored by the modern pharmaceutical industry, despite the fact that lichens produce a large member of low molecular weight molecules with diverse structures and that studies have provided evidence of biological activity in extracts from whole lichens (Table-1). There are two contributing reasons for this; (1) lichens are slow growing in are and (2) they are difficult to propagate and resynthesize in culture (Ahmadjian, 1993; The Lichen Symbiosis, Blaisdell Publishing Company). Industrial scale harvests are neither ecologically sensible nor sustainable and for many species are not feasible. Even if the lichen cultures are established in-vito they do not produce the typical lichen substances and the techniques to encourage this are still unknown.

TABLE 1
Previously described bioactive constituents from different Lichens.

| | | | |
|---|---|---|---|
| BiologicaI Activity | Lichen Substance | Origin | Reference |

# Enzyme Inhibition

| | | | |
|---|---|---|---|
| Monoamine oxidase | Norsolorinic acid | Solorina crocea | Okuyama et al 1991 |
| inhibition | | | |
| | Confluentic & 2′-0- | Higher plant | Endo et al. 1994 |
| | methylperlatolic acids | (himatanthus | |
| | | succuuba) | |
| Prostaglandin | Metadepsides | | Sankawaetal 1982 |
| biosynthesis | | | |
| inhibition | | | |
| Trypsin inhibition | Atranorin | Pseudevernia | Proksa et al 1994 |
| | | furfuracea | |
| Tyrosinase inhibition | Resorcinol deriv. | Protousnea spp. | Kinoshita et al 1994 |
| # Animal Assay | | | |
| Analgesic and | Diffractaic & usnic acids | Usnea diffracta | Okuyama et al 1995 |
| antipyretic | | | |
| Anti-inflammatory | Diffractaic & usnic acids | Usnea diffracta | Otsuka et al 1972 |
| Anti-melanin | Resorcinol deriv. | organic synthesis | Matsubara et al 1998 |
| biosynthesis | | | |
| Anti-tumor cell | (−)-Usnic acid | Cladonia leptoclada | Kupchan & |
| | | | Kopperman 1975 |
| | Usnic acid deriv. | Organic synthesis | Takai et al. 1979 |
| | Polysaccharide (GE-3) | Umbilicaria | Fukuoka et al 1968 |
| | | esculenta | |
| *Ishikawa cells | Usnic acid | | Cardarelli et al 1997 |
| *Melanoma B-16 | Cristazarin | Caldonia cristatella | Yamamoto et al 1998 |
| cells | | | |
| | Resorcinol deriv. | organic synthesis | Matsubara et al 1998 |
| Auto-oxidation | 1′-Chloropannarin & | Erioderma chielense | Hidalgo et al. 1994 |
| inhibition | Pannarin | | |
| (Antioxidant) | | | |
| Cholesterol synthesis | Gyrophoric acid deriv. | Umbilicaria | Kim 1982 |
| inhibition | | esculenta | |
| Insect-growth | Atranorin & vulpinic acid | Umbilicaria | Slansky 1979 |
| inhibition | esculenta | | |
| | Caperatic acid | Cetraria oakesia | Lawrey 1983 |
| Long-term | Polysaccharide (PC-2) | Flavoparmelia | Smriga et al 1998 |
| potentiation | | caperata | |
| enhancement | | | |
| Nematocidal | Orsellinic acid deriv. | Evernia prunastri | Ahad et al 1991 |
| # Plant Assay | | | |
| Mitosis inhibition in | Retigeranic acid | Lobaria retigera | Reddy et al 1978 |
| root tips | | | |
| Moss germination | Evernic & squamatic | Cladonia squamosa | Lawrey 1977 |
| inhibition | acids | | |
| Photosystem II | Usinic acid | | Inoue et al 1987 |
| inhibition | | | |
| | Depsides | Usnea longissima etc | Endo et al 1998 |
| Plant-growth | Depsides | Usnea longissima | Nishitoba et al 1987 |
| inhibition | | | |
| | Usnic acid | Cladonia substellata | Yano-Melo et al |
| | | | 1999a |
| | Fumarprotocetraric acid | Cladonia verticillaris | Yano-Melo et al |
| | | | 1999b |
| Plant cell-growth, | Usnic acid | | Cardarelli et al 1997 |
| seed germination | | | |
| inhibition & | | | |
| protoplast viability | | | |

# Microorganism Assay

| | | | |
|---|---|---|---|
| (a) Anti-viral | Polysaccharide (GE-3S) | Umbilicaria | Hirabayashi et al 1989 |
| Anti-HIV | | esculenta | |
| HIV-l Integrase | Depsides & desidones | | Neamati et al (1997) |
| inhibition | | | |
| Anti-HSV-l | Hypericin deriv. | Nephroma | Cohen et al 1996 |
| | | laevigatum | |
| Epstein-Barr virus | Lichesterinic, (+)-usnic, | Usnea longissima | Yamamoto et al 1995 |
| activation inhibition | (−)-usnic & evernic acids | | |
| (b) Anti-bacteria | Vulpinic, (+)- & (−)-usnic | | Lauterwein et al 1995 |
| *Enterococcus | acids | | |
| faecaolis & E. faeciem | | | |
| Bacillus subtilis, | Atranol | Stereocaulon | Caccamese et al 1986 |
| E.coli | | vesuvianum | |
| *Helicobacter pylori | Protolichesterinic acid | Cetraria islandica | Ingolfsdottir et al |
| | | | 1997 |
| *Mycobaterium | Depsides & usnic acid | Cladonia crispatula | Pereira et al 1997 |
| smegmatis | | | |
| *Staphlycoccus | Alectrosarmentin | Alectoria sarmentosa | Gollapudi et al 1998 |
| aureus | | | |
| | Cristazarin | Cladonia cristatella | Yamamoto et al 1998 |
| | Decarboxystenosporic | Usnea diffracta | Yamamoto et al 1998 |
| | acid | | |
| *Leishmania chagasi | Atranorine & difractaric | | Jota et al |
| | acid | | |
| (c) Anti-fungal | Methyl haenatommate | Stereocaulon | Hickey et al 1990 |
| | | ramulosum | |
| | Vulpinic, (+)- & (−)-usnic | Alectoria ochroleuca | Lauterwein et al 1995 |
| | acids | | Proksa et al 1996 |
| | (−)-Usnic acid deriv. | | |
| Saccharomyces | Atranol | Stereocaulon | Caccamese et al 1986 |
| cerevisiae | | vesuvianum | |
| P. digitatum, | Methyl β- | Parmelia furfuracea | Caccamese et al 1985 |
| S. cerevisiae | orcinolcarboxylate | | |
| *Fusarium | Usnic acid | | Cardarelli et al 1997 |
| moniliforme | | | |
| # Anti-insect | | | |
| | atranorin and vulpinic- | | Slansky, (1979) |
| Spodoptera | acid | | Emmerich, et al. |
| ornithogalli | | | (1993) |
| Spodoptera littoralis | | | |

It is thought that most secondary metabolites of lichens are made by the mycobionts (Huneck, and Yoshimura, (1996) Identification on of Lichen Substances, Springer-Verlag). This is not surprising because final compounds are well known in medicine (e.g. peniciliin and cyclosporin). It is possible, however, that the photobionts also contribute to the repertoire of lichen metabolites. Cyanobacteria produce many bio-active secondary metabolites (Namikoshi, M. and Rinehat, K. L. (1996) Bioactive compounds produced by cyanobacteria. J. Ind. Microbiol. 17, 373-143) and there is an example of a patented anti fungal compound produced by a strain of Nostoc isolated from a lichen (U.S. Pat. No. 4,946,835, Merck & Co).

There are compelling reasons for compounding the search for natural-product drugs because previously reliable standard antibiotics are becoming less and less effective against new strains of multi drug-resistant pathogens. It has even been suggested that the end of the antibiotic era is fit approaching. In the past, search for pharmaceutically active molecules concentrated on the products of microbes that can be cultivated in the laboratory. More recently synthetic chemical methodologies have attracted a great deal of attention and combinatorial chemistry as been promoted as a source of molecules for automated high-throughput screening methods. Although these approaches have provided some lead molecules there is still a great need to discover novel chemical eyes for therapeutic use.

Systemic and superficial fungal infections affect millions of people throughout the world. Most of these diseases are caused by Candida albicans, Cryptococcus neoformans, Aspergilhus sp., Trichophyton sp., Microsporum gypseum, Epidermophyton floccossum that are infectious in nature. In India, large number of people are involved in agriculture with majority of them living in villages where due to the prevailing unhygienic conditions the incidence of mycotic infections are severe. Fungal infections are also assuming increasing importance on account of decrease in immune Systems mainly because of organ transplant operations, cancer chemotherapy and acquired immune deficiency syndrome (AIDS). Moreover the skin infections spread rapidly due to poor hygienic conditions and over population as well as increasing level of environmental pollution. To counter these infections only a handful of anti fungal agents such as greseofulvine, amphotericin and nystatin are available in the market, although the available antibacterials are replete. Most of these antifungals are synthetic derives with ham side effects to human and animals. Compounding this problem is the development of resistance towards commonly used drugs thus rendering the chemotherapy less useful. Therefore new antifungal substances from natural sources have to be generated to counter the resistance phenomenon During 1990-96 the world market for animals was over US $ 1500 millions representing 1.5% of the total global anti-infective market. Currently anti-fungals (both topical and systemic) represent more than 6% of the total anti-infective agents. The world market for antifungals is expanding at the rate of 20% per annum and is estimated to reach over US $ 600 million/annum. However, many of the synthetic drugs produce side effects in immune stressed individuals. On the other hand natural products and their formulations made out of herbal sources will have more acceptances than the synthetic antifungals.

### OBJECTS OF THE INVENTION

The main object of the present invention to identify Lichen extract, which can specifically kill the polyene drug resistant fungal infections of humans.

It is also the object of the invention to isolate, characterize and establish the nature of the bioactive molecule from the active lichen ea by bioactivity-guided fractionation.

Still another object of the invention is to test the ergosterol binding ability of the bioactive molecule using in-vitro assays.

### SUMMARY OF THE INVENTION

Accordingly the present invention provides an a gal/anticancer composition comprising a pharmaceutically effective amount of methyl-o-orcinol carbonate of formula I and a pharmaceutically acceptable carrier

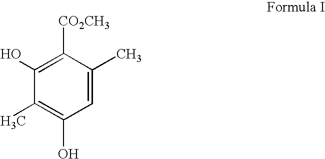

In one embodiment of the invention, the composition is anti-fungal and the methyl-β-orcinol carboxylate of formula I is present in a concentration in the range of 10-400 μg/ml.

In another embodiment of the invention, the composition is anticancer and the methyl-β-orcinol carboxylate of formula I is present in concentration in range of 1-10 μg/ml.

In another embodiment of the invention, the fungus is from the group of yeasts comprising of Candida sp, exemplified by Candida albicans.

In another embodiment of the invention, the cancer is liver, colon, ovarian or mouth (oral) cancer of humans.

The invention also relates to a method of treatment of fungal infections in a subject comprising administering to the subject an anti-fungal composition comprising a pharmacy effective amount of methyl-β-orcinol carboxylate of formula I and a pharmaceutically acceptable carrier.

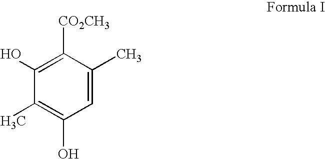

In one embodiment of the invention, the methyl-β-orcinol carboxylate of formula I is isolated from lichen Everniastrum cirrhatum.

In another embodiment of the invention, the fungus comprises a multiple or single drug resistant strain.

In another embodiment of the invention, the methyl-β-orcinol carboxylate of formula I is present in a concentration in the range of 10-400 μg/ml.

In a further embodiment of the invention, the fungus is from the group of yeasts comprise of Candida sp, exemplified by Candida albicans.

In a further embodiment of the invention, the fungus is a polyene drug resistant strain, the polyene drug being exemplified by nystatin and anphotericin

In yet another embodiment of the invention, the fungus comprises an azole resistant strain, the azole drug being exemplified by clotrimazole, flucanoazole, itracanoazole and micanazole.

In yet another embodiment of the invention, the fungus is simultaneously resistant to both polyene and azole classes of antibiotics.

The subject is preferably human.

The present invention also provides a method for the treatment of cancer in a subject such as a human being, the cancer being either of liver, colon, ovarian and mouth (oral) cancer comprising administering to the subject a pharmaceutically effective amount of methyl-β-orcinol carboxylate of formula I and a pharmaceutically acceptable carrier.

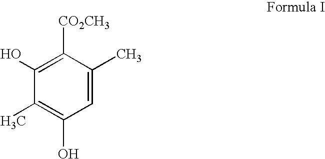

In one embodiment of the invention, the concentration of methyl-β-orcinol carboxylate of formula I is in the range of 1-10 μg/ml.

The present invention also relates to the use of methyl-β-orcinol carboxylate of formula I 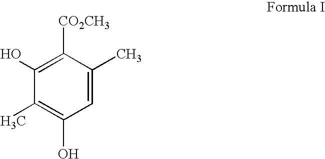
for the treatment of fungal infection or cancer in a subject.

### DETAILED DESCRIPTION OF THE INVENTION

The present invention relates to the use of a biomolecule methyl-β-orcinolcarboxylate of formula I isolated form a lichen (Everniastrum cirrhatum), 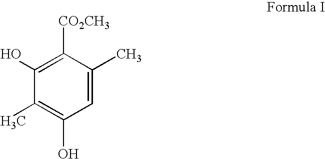
for treat pathogenic fungal infections of humans that are resistant to polyene antibiotics such as amphotericin B, nystatin etc. However, the biomolecule does not possess ergosterol-binding property.

Infections due to Candida sp account for about 80% of all major systemic fungal infections Candida is now the fourth most prevalent organism found in bloodstream infections and is the most common cause of fungal infections in immuno-compromised people. Vaginal candidiasis commonly affects women, including those with normal immunity, especially after antibiotic use.

Lichens were collected from Narayan Ashram, Pithoragarh; Uttaranchal, India in the month of April 2002. Subsequently the lichens were identified taxonomically as Everniastrum cirrhatum. The collected lichen was air dried in shade and ground to fine powder. The powdered lichen material was used flier for chemical analysis. Ethanol extract was prepared and tested against Candida albicans MTCC 1637 (equivalent to ATCC 18804) the fungi that cause different forms of candidiasis in humans and drug rent mutants of the fungi. Amphotericin and nystatin are standard polyene antifungal drugs used in chemotherapy. Candida albicans isolates resistant to these polyene antibiotics are already reported. High-level residence to amphotericin B, seen in all the major Candida species, is most common in neutropenic patients who have received prolonged courses of amphotericin B. Such drug resistant infections are clinically difficult to treat and are physician's nine Hence, we developed such polyene resistant strains of Candida albicans in-vitro and evaluated the anti-admiral effect of lichen exacts/compounds against them. The extract and subsequent solvent (hexane and ethyl acetate) fractions were found to be active against amphotericin and nystatin resistant Candida. Bioactivity guided fractionation of the active fractions resulted in the isolate of active compounds by column chromatography. The active compound could be crystallized from 96% hexane; 4% ethyl acetate fraction. The purified compound was analyzed by spectroscopic techniques using ¹H & ¹³C NMR, LC-MS etc to decipher the chemical structure. Compound was identified as methyl-β-orcinolcarboxylate, of formula I. The compound is a colorless crystal with melting temperature of 137° C. Caccamese et al (1985) have already found that the methyl-β-orcinolcarboxylate inhibit the growth of yeast strains such as Saccaharomyces cerevisiae. However, in this study we shown a unique property of methyl-β-orcinolcarboxylate wherein the compound specifically inhibits the growth of polyene and azole drug resistant strains of Candida albicans and Saccaharomyces cerevisiae. The principal sterol in the fungal cytoplasmic membrane, is the target site of action of amphotericin B and the azoles. Amphotericin B, a polyene, binds irreversibly to ergosterol, resulting in disruption of membrane integrity and ultimately cell death. Therefore, the ability of lichen compounds to bind to ergosterol was also investigated using in-vitro ergosterol binding assay (Antonio & Molinski 1993; J. Natl. Prod. 56:54-61). The results indicated that the compounds do not possess any specificity to ergosterol in the wild type and drug resistant strains of Candida sp.

The present invention therefore provides an antifungal/anticancer composition comprising a pharmaceutically effective amount of methyl-β-orcinol carboxylate of formula I and a pharmaceutically acceptable carrier. A concentration of methyl-β-orcinol carboxylate of formula I in the range of 10-400 μg/ml provides antifungal activity against the group of yeasts comparing of Candida sp, exemplified by Candida albicans. A concentration of methyl-β-orcinol carboxylate of formula I in the range of 1-10 μg/ml provides anticancer activity against liver, colon, ova or mouth (oral) cancer of humans.

The methyl-β-orcinol carboxylate of formula I is isolated from lichen Everniastrum cirrhatum.

The fungus can be either a multiple drug resistant or single drug resistant strain For example, the fungus can be from the group of yeasts comprising of Candida sp, exemplified by Candida albicans. The drugs in question can be a polyene drug exemplified by nystatin and anphotericin or a azole drug exemplified by clotrimazole, flucanoazole, itracanoazole and mica azole.

The following examples are inactive and should not be construed as limiting the scope of the invention in any manner.

### EXAMPLES

1 Violation of Polyene Drug Resistant Mutant Strains of Candida albicans MTCC 1637 (Equivalent to ATCC 18804)

C. albicans was grown to log phase in Sabouraud's dextrose broth (5 ml) for 48 hrs at 37° C. in a shaker at 250 rpm. The cells were pelleted by centrifugation at 500 rpm at 4° C. and the pellet was dissolved in 5 ml phosphate buffered sale PBS (6.8 pH). The culture was divided in to five groups of 1 ml each in eppendrof tubes.

Ethyl methane sulfonate (EMS) was added to each of the culture tube @ 0.1% (v/v) and allowed to grow for 40 min. Then the mutagen was completely washed off thrice by repeatedly pelleting the cells and re-dissolving in PBS. The mutagenized stocks was then diluted in Sabouraud's dexose broth two folds and allowed to grow for 6 hrs at 37° C. in a shaker at 250 rpm Titre of the cells before treatment with EMS and immediately after treatment with EMS was recalculated to obtain the killing percentage in each of the five tubes. The mutagenized and fixed cultures were them plated in Sabouraud's dextrose agar containing different concentration of amphotericin, nystatin and clotrimazole.

The colonies found growing after 5^{th} day from each of the five mutagenized stocks were then purified thrice separately by streaking in the same medium containing the antibiotics.

2. Drug Resistance of Mutant Strains Against Polyenes and Azoles

The drug resistance property of the mutants was studied by standardized disc diffusion assay (Bauer at al 1966, American Journal of Clinical Pathology 45: 493-496) with slight modifications. The discs were prepared (5 mm diameter made of Whatman #3 filter paper) by impregnating 8 μl of test compound and placing them on pre-inoculated agar surface.

A disc containing only the solvent was used a the control. A zone of growth inhibition surrounding the disc is indicative of the resistant nature of the s s to antibiotics. As is evident from this sample the results indicate that all the mutant strains were highly resistant to amphotericin ad nystatin as the zone of growth inhibition was far less in mutants than that of the wild type parent strain However only Amph C7R, Amph C6R Clo 31R and Clo 28R were only resistant to clotrimazole indicating of less zone of growth inhibition.

TABLE 2

| | Amphotericin | Nystatin | Clotrimazole |
|---|---|---|---|
| Yeast strains | 80 μg/disc | 80 μg/disc | 80 μg/disc |
| Candida albicans | 9 | 22 | 17 |
| MTCC | | | |
| (Wild Type) | | | |
| Amph A8R | — | 3 | 22 |
| Amph C7R | — | 2 | 12 |
| Amph C6R | — | 4 | 10 |
| Amph D1R | — | 4 | 27 |
| Amph 100R | 2 | 13 | 25 |
| NYS 4R | 2 | 8 | 20 |
| NYS 26R | 4 | 9 | 19 |
| Clo 31R | 6 | 17 | 11 |
| Clo 28R | 12 | 21 | 14 |

3. Collection and Traction of Lichen Materials:

Two kg of the lichen (Everniastrum cirrhatum) material were collected from Narayan Ashram, Pithoragarh, Uttaranchal, during the month of April 2002. They were separated and air-dried at room temperature (35° C.-40° C.) in shade. After air drying they were ground and sieved to fine powder in a mixer grinder. 1.5 kg of the powdered materials were dipped in absolute ethanol in a percolator for 72 hrs at room temperature (35° C.-40° C.).

Ethanol extract was filtered using Whatman filter paper No. 1 and concentrated at the 60° C. under reduced pressure. The ethanolic extract was then lyophilized to obtain 15.5 g of crude extract. Stock of 100 mg/ml was made in DMSO and tested for bio-activity.

4. Bioactivity Guided Fractionation of the Lichen Materials

Solvent fractionation of the active crude extracts was undertaken to isolate the active principle. Ethanolic extract was dissolved in 500 ml of hexane. Then it was filtered using Whatman No. 1 filter paper. The insoluble portion was dissolved in 500 ml of ethyl acetate. All the solvent fractions were concentrated at 40° C. under reduced pressure to obtain 3 g of hexane and 1.5 g of ethyl acetate extract and tested. The results indicate that both ethyl acetate and hexane fraction obtained from the crude extract possessed the bioactivity against drug resistant strain of C. albicans. The hexane fraction was con siderably more active than the ethyl acetate extract.

TABLE 3

| | Net Zone of growth inhibition (mm) | | |
|---|---|---|---|
| | Crude | | |
| | ethanolic | | Ethyl acetate |
| | lichen extract | Hexane Frac | Frac. |
| Yeast strains | 800 μg/disc | 800 μg/disc | 800 μg/disc |
| Candida albicans | 4 | 6 | — |
| MTCC (WT) | | | |
| Amph A8R | 11 | 13 | 7 |
| Amph C7R | 9 | 13 | 5 |
| Amph C6R | 10 | 14 | 8 |
| Amph D1R | 12 | 15 | 7 |
| Amph B1R | 8 | 11 | 4 |
| NYS 4R | 10 | 14 | 4 |
| NYS 26R | 11 | 18 | 10 |
| Clo 3lR | 7 | 5 | 2 |
| Clo 28R | 7 | 10 | 5 |

5. Purification and Characterization of the Active Molecule

The hexane and ethyl acetate fractions thus obtained are mixed together and further fractionated in a glass column having an internal diameter of 3.0 cm and leg of 72.0 cm. Hexane was used as the initial mobile base and silica gel (particle size 60-120 mesh) as the stationary phase. Different fronts of approximately 100 ml were collected and dried under vacuum Concentrated fractions were then run on TLC plates and fractions of similar TLC pattern were pooled together. After about 3 liter of hexane faction collected the polarity of the mobile phase was slightly increased from fraction No. 36 by adding ethyl acetate to hexane (4% of ethyl acetate in final volume). Similarly fractions No. 64 to 78 were combined together based on identical 7-spot bands as appeared in TLC. Above fractions were dissolved in 50 ml of acetone and kept at room temperature (25-30° C.) for crystallization of compounds. Crystals thus obtained were again properly washed with acetone and TLC of crystals was carried out by using a mobile phase of benzene 98% plus acetone 2%. TLC plates showed a singe spot on exposing to iodine fume. These TLC plates exhibited a single dark red colored spot when dipped in bacopa reagent (vale 3.5 g, H₂SO₄ 17.8 g, absolute alcohol 332.5 ml) and heated at 120° C. for 5 minutes. About 40 mg of the crystal could be collected from the above run. The melting temperature of crystal thus obtained was found to be 137° C.

The active spot obtained by TLC was further purified by repetitive column chromatography, which can be performed by a person skilled in the art and the analyzed by ¹H & ¹³C NMR, LC-MS to determine the structure of the active pure compound. On the basis of spectroscopic data the compound isolated was identified as Methyl-β-orcinolcarboxylate.

6. Specific Anticandidial Activity of Methyl-β-Orcinolcarboxylate Acid Against Polyene and Azole Resistant Strains

The pure compound isolated was then tested against polyene and azole resistant strains of Candida albicans. The data described below indicates that the compound methyl-β-orsellinic acid was able to inhibit the growth of drug resistant strains in a dose dependant manner whereas it was inactive against the wild type strain. In another experiment well-defined amphotericin and nystatin resist strains of Saccaharomyces cerevisiae were used in the assay. These streams designated as erg 2 and erg 6 carry mutations in the ergosterol biosynthetic pathway and therefore are unable to synthesize ergosterol which are the binding site of polyene drugs. Therefore absence of ergosterol results in polyene resistance. The results suggests that methyl-β-orcinolcarboxylate was able to specifically inhibit the growth of polyene drug resistant Saccaharomyces cerevisiae.

TABLE 4

| | Net zone of growth inhibition (mm) | | |
|---|---|---|---|
| | produced by methyl-β- | | |
| | orcinolcarboxylate | | |
| Yeast strains | 40 μg/disc | 80 μg/disc | 320 μg/disc |
| Candida | — | — | — |
| albicans | | | |
| MTCC (WT) | | | |
| Amph A8R | 1 | 2 | 4 |
| Amph C7R | 2 | 5 | 7 |
| Amph C6R | 2 | 4 | 5 |
| Amph D1R | — | 4 | 6 |
| Amph B1R | 2 | 3 | 4 |
| NYS 4R | 2 | 7 | 8 |
| NYS 26R | 2 | 6 | 8 |
| Clo 31R | — | 3 | 4 |
| Clo 28R | — | 4 | 6 |

TABLE 5

| | Net zone of growth inhibition (mm) | | | | | |
|---|---|---|---|---|---|---|
| | | | | Methyl | | |
| | Lichen | | Ethyl | β- | | |
| | crude | Hexane | acetate | orcinolc | | |
| | extract | Frac | Frac. | arboxylate | Amphotericin | Nystatin |
| Yeast strains | 800 μg/disc | 800 μg/disc | 800 μg/disc | 80 μg/disc | 80 μg/disc | 80 μg/disc |
| Saccharomyces | — | 3 | — | 2 | 8 | 23 |
| Cerevisiae | | | | | | |
| ABC 287 | | | | | | |
| (WT) | | | | | | |
| erg 2 | 7 | 10 | 5 | 6 | 6 | 16 |
| erg 6 | 10 | 17 | 5 | 6 | 5 | 13 |

7. Anticancer Property of Methyl β-Orsellinic Acid Against Human Cancer Cell Lines

Cytotocity testing in vitro was done by the method of Woerdenbag et al., 1993; J. Nat. Prod. 56 (6): 849-856). 2×10³ cell were incubated in the 5% CO₂ incubator for 24 h to enable them to adhere properly to the 96 well polystyrene microplate (Grenier, Germany). Test compounds dissolved in 100% DMSO, Merx:,Germany) in at least five doses were added and left for 6 h after which the compound plus media was replaced with fresh media and the cells were incubated for another 48 h in the CO₂ incubator at 37° C. The concentration of DMSO used in our pediments an exceeded 1.25%, which was found to be non-toxic to cells. Then, 10 μl MTT [3-(4,5-dimethylthiazol-2-yl)-2,5diphenyltetrazolium bromide; Sigma M 2128] was added, and plates were incubated at 37° C. for 4 h. 100 μl dimethyl sulfoxide (DMSO, Merck, Germany) were added to all wells and mixed thoroughly to dissolve till dark blue crystal. After a few influxes at room temperature to ensure that all crystals were dissolved, the plates were read on a SpectraMax 190 Microplate Elisa reader (Moecular Devices Inc., USA), at 570 nm. Plates were normally read within 1 h of adding the DMSO. The experiment was done in triplicate and the inhibitory concentration (IC) values were calculated as follows: % inhibition=[1−OD (570 am) of sample well/OD (570 nm) of control well]×100. IC₉₀ is the convention μg/mL required for 90% inhibition of cell growth as compared to that of untreated control. The results described indicate that the ethanolic crude extract of the lichen and the isolated pure compound methyl-β-orcinolcarboxylate was active against liver (WRL-68); colon (Caco-2); ovarian (MCF-7 & PA-1) and oral (KB 403) human cancer cell lines.

TABLE 6

| | WRL-68 | | MCF-7 | | PA-1 | | Caco2 | | KB-403 | |
|---|---|---|---|---|---|---|---|---|---|---|
| Lichen | IC- | IC- | IC- | IC- | | IC- | IC- | IC- | IC- | IC- |
| compounds | 50 | 90 | 50 | 90 | IC-50 | 90 | 50 | 90 | 50 | 90 |
| Crude | 0.07 | — | 0.05 | >10 | 0.5 | — | — | — | 0.25 | — |
| ethanolic | | | | | | | | | | |
| extract | | | | | | | | | | |
| Methyl-β- | 1.0 | 5.0 | 1.0 | 5.5 | 0.025 | 4.0 | 1.5 | 3.5 | 0.04 | 4.5 |
| orcinol- | | | | | | | | | | |
| carboxy | | | | | | | | | | |
| late | | | | | | | | | | |

* Data given as μg/ml.

### REFERENCES

Ahad, A. M., Goto, Y., Kiuchi F., Tsuda, Y. and Sato, T. (1991) Nematoidal principles in “Oakmoss absolute and nematocidal activity of 2,4-aydroxybezoates. Chem. Pharm. Bull 39, 1043-1046.

Caccamese S. R M Toscano, F. Bellesia and A Pinetti (1985). Methyl-b-orcinolcarboxylate and depsides from Parmelia furfuracea. J. Nat. Prod. 48: 157-158.

Cardareli, M., Scino, G., Campanela, L., Ercole, P., Nardone, F. De C., Alesiai, O. and Rossielo, F. (1997) Antimitotic effects of usnic acid on different biological systems. Cell Mol. Life Sci. 53, 667-672.

Cohen, P. A, Hudson, J. B. and Towers, G. H. N. (1996) Antiviral activities of anthraquinones, bianthrones and hypericin derivatives from lichens. Experientia 52, 180-183.

Endo, T., Takahads T., Kinosbita, Y., Yamamoto, Y. and Fumihiko Sato, F. (1998) Inhibition of photosystem II of spinach by lichen derived depsides. Biosci. Biotechnol. Biochem. 62, 2023-2027.

Endo, Y., Hayashi, R, Sato, T., Maruo, M., Oha, T. and Nozoe S. (1994) Confluentic acid and 2′-O-methylperlatolic acid, monoamine oxidase B inbitors in a Brazilian plant, Himatanthus sucuuba. Chem. Pharm. Bull 42, 1198-1201.

Fukuoka, F., Nakanishi M., Shibata, S., Nishikawa, Y., Takeda, T. and Tanaa, M. (1968) Polysaccharides in lichens and fungi. Antitumor activity on Sarcoma-180 of the polysaccharide preparations from Gyrophora esculenta, Cetraria islandica orientalis:Gann 59,421-432.

Fitzsimmons, B. J.; Rokah, J. In Leukotrienes and Lippoxygenases; Rokach, J., Ed.; Elsevier: Amsterdam, 1989; pp 427-502.

Gollapudi, S. A., Toiikepalii, H., Jampani, H. B., Mirhom, Y. W., Drake, S. D., Bhattiprolu, K R, Velde, D, V. and Mitscher, L. A. (1994) Alectrosarmentin, a new antimicrobial dibenzofuranoid lactol from the lichen Alectoria sarmentosa J. Natural Prod. 57, 934-938.

Gissurarson, S. R; Sigurdsson, S. B.; Wagner, H.; Ingolfsdottier, K. J. Pharmacol. Exp Ther 1997, 280,770-773.

Hickey, B. J., Lumsden, A. J., Cole, A. L. J. and Walker, J. R. L. (1990) Antibiotic compounds from New Zealand plants: methyl hamatote, an anti-fungal agent from Stereocaulon ramulosum. New Zealand Natural Sci. 17, 49-53.

Hidalgo, M. E., Fernandez E., Quilhot, E. and Lissi E. (1994) Antioxidanactivity of depsides and depaidons. Phytochemistry 37, 1585-1587.

Hirabayashj L., Iehata, S., Ito, M., Shigeta, S., Narui, T. and Shibata, S. (1989) Inhibitory effect of a lichen polysaccharide sulfate, GE-3-S, on the reparation of human immunodeficiency virus (HIV) in vitro. Chem. Pharm. Bull. 37, 2410-2412.

Ingolfsdottier, K, Hjalmardottir, M. A, Sigurdsson, A, Gudjonsdottir, G. A, Brynjolfsdottir, A. and Steingrimsson, O. (1997) In vitro susceptibility of Helicobacter pylori to protolichesteic acid from the lichen Cetraria islandica. Antimicrobial Agents & Chemotherapy 41, 215-217.

Inoue, H, Noguchi, M. and Kubo, K. (1987) Site of inhibition of usnic acid at oxidizing side of photosystem 2 of spinach chloroplast. Photosynthetica 21, 88-90.

Kim, C. H. (1982) Studies on the substances contained in Gyrophora esculenta lowering plasma and liver cholesterol levels Part 4. J. Iwate Med. Ass. 34, 669-672.

Kinoshita, K, Matsubara, H, Koyama, K, Takahashi, K, Youhimura, I., Yamamoto, Y., Higuchi, M., Miura, Y., Kinoshita, K and Kawai, K. (1994) New phenolics from Protousnea species. J. Hattori Bot. Lab. 75, 359-364.

Kupchan, S. M. and Kopperman, H. L. (1975) 1-Usnic acid: tumor inhibitor isolated from lichens. Experientia 31, 625-626.

Lauterwein, M., Oethinger, M., Belsner, K, Peters, T. and Marre, R. (1995) In vitro activities of tie lichen secondary metabolites vulpinic acid, (+)-usnic acid, and (−)-usnic acid against aerobic and anaerobic microorgaisms. Antimicrobial Agent Chemotherapy 39, 2541-2543.

Lawrey, J. D. (1977) Adaptive significance of O-methylated lichen depsides and depsidones. Lichenologist 9, 137-142.

Lawrey, J. D. (1983) Vulpinic and plastic acids as lichen antiherbivore compounds: contrary evidence. Bryologist 86, 365-369.

Matsubara, H., Kinoshita, K., Koyama, K, Yang Ye, Takahashi, K., Yoshimura, I., Yamamoto, Y., Miura, Y., and Kinoshita Y. (1997) Anti-tyrosinae activity of lichen metabolites and their synthetic analogues. J. Hattori Bot. Lab. 83, 179-185.

Matsubara, H., Koshita, K, Koyama, K, Yang Ye, Takuhashi, K., Yoshiura, I., Yamamoto, Y., Miura, Y., and Kinoshita, Y. (1997) Inhibitory effect of lichen metabolites and their synthetic analogues on melanin biosynthesis in cultured B-16 melanoma cells. Natural Product Sci. 4, 161-69.

Nishitoba, Y., Nishimura, H, Nishiyama, T. and Mizutani, J. (1987) lichen acids, plant growth inhibitors from Usnea longissima. Phytochemistry 26, 3181-3185.

Ogmundsdottir, H., M; Zoega, G. M.; Gissurarson, S. R.; Ingolfsdottier, K. J. Pharm. Pharmacol. 1998, 50,107-115.

Otsuka, H., Komiya, T., Tsukumi, M., Toyosato, T. and Fujimura, H. (1972) Studies on anti-inflammatory drugs. Anti-inflammatory activity of crude drugs and plants. (II). J. Takeda Res. Lab. 31, 247-254.

Okuyana, B., Hossain, C. F. and Y M. (1991) Monoamine oxidase inhibitors from a lichen, Solorina crocea (L.) ACH. Shoyakugaku Zasshi 45, 159-162.

Okuyama, B.; Umeyama, K; Yamazaki, M.; Kinoshita, Y.; Yamamoto, Y. Planta Med. 1995, 61,113-115.

Okuyama, B., Umeyama, K., Yamazaki, M., Kinoshita, Y. and Yamamoto, Y. (1994) Usnic acid and diffactaic acid as angesic and antipyretic components of Usnea diffracta Vain. Planta Medica 61, 113-115.

Pereira, E. C., da Silva, N. H., de Campostakaki, G. M., Xavier-Filho, L., Legaz, M. E. and Vicente, C. (1997) Antimicrobial activity of biologically active compounds from the lichen Cladonia crispatula. Boletin Ecotropica Ecosistemas Tropicales 31, 10-19.

Prolsa, B., Adamcova, J., Sturdikova, M., and Fuska, J. (1994) Metabolites of Pseudevernia furfuracea (L.) Zopf. and their Inhibition potential of prototypic enzymes. Pharmazie 49, 282-283.

Proksa, B., Sturdikova, M., Pronayova, N. and Liptaj, T. (1996) (−)-Usnic acid and its derivatives. Their inhibition of fungal growth and enzyme activity. Pharmazie 51, 195-196.

Reddy, P. D., Rao, P. S. and □@Subramanyam, S. (1978) Influence of some lichen substances on mitosis in Allium cepa root tips. Indian J. Exp. Biol. 16, 1019-1021.

Sankawa, U., Shibuya, m, Ebizuka, Y., Noguchi, H., Koshita, T. and Iitaka, Y. (1982) Depsides as potent inhibitor of prostaglandin biosynthesis: a new active site model for fatty acid cyclooxygenase. Prostaglandin 24, 21-34.

Slansky, F. Jr. (1979) Effect of the lichen chemicals atranorin and vulpinic acid upon feeding and growth of larvae of the yellow-striped armyworm, Spodoptera ornithogalli. Environ. Entomol. 8, 865.

Smriga, M., Saito, H., Shibata, S., Narui, T., Okuyama, T. and Nishiyama, N. (1996) PC-2, linear homoglucan with f_{L}-linkages, peripherally enhances the hippocampal long-term potentiation. Pharmaceutical Research 13, 1322-1326.

Takai, M., Uehara, Y. and Beisler, J. A. (1972) Usnic acid derivatives as potential antineoplastic agents. J. Med. Chem. 22, 1380-1384.

Yamamoto, Y., Kinoshita, Y., Matsubara, H., Kinoshita, K, Koyama, K, Takahashi, K., Kurokawa, T. and Yoshimura I. (1998) Screening of biological activities and isolation of biological-active compounds from lichens. Recent Res. in Phytochme. 2, 23-34.

Yammoto, Y., Miura, Y., Kinoshita, Y., Higuchi, M., Yamada, Y., Murakami, A, Ohigashi, H. and Koshimizu, K. (1995) Screening tests of tissue cultures and thalli of lichens toward the inhibition of Epstein-Barr virus activation and some of their active constitutes. Chem. Pharm. Bull. 43, 1388-1390.

Yano-Melo, A. Y., Vicente, C. and Xavier-Filhoh, L. (1999a) Influences of Cladonia sunstellata Vainio acts and usnic acid on germination and growth of Allium cepa L. seedlings. Tropical Bryology 16, 11-15.

Yano-Melo, A. Y., Vicente, C. and Xavier-Filhoh, L. (1999b) Allelopaiic defect of the Cladonia verticillaris lichen extracts and fumarprotocetraric acid on the early growth of germinated seedlings in Allium cepa L. Tropical Bryology 17,133-139.

1-6. (canceled)

7. A method for the treatment of fungal infections in a subject comprising administering to the subject an anti-fungal composition comprising a pharmaceutically effective amount of methyl-β-orcinol carboxylate of formula I and a pharmaceutically acceptable carrier.

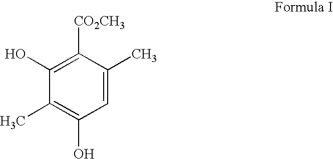

8. A method as claimed in claim 7, wherein the methyl-β-orcinol carboxylate of formula I is isolated form lichen Everniastrum cirrhatum.

9. A method as claimed in claim 7, wherein the fungus comprises a drug resistant strain.

10. A method as claimed in claim 7, wherein the methyl-β-orcinol carboxylate of formula I is present in a concentration in the range of 10-400 μg/ml.

11. A method as claimed in claim 9, wherein fungus is from the group of yeasts comprising of Candida sp.

12. A method as claimed in claim 11, wherein the fungus is Candida albicans.

13. A method as claimed in claim 9, wherein fungus is a multiple/single drug resistant strain.

14. A method as claimed in claim 13, wherein the fungus is a polyene drug resistant strain.

15. A method as claimed in claim 14, wherein the polyene drug is nystatin or anphotericin.

16. A method as claimed in claim 13, wherein the fungus comprises an azole resistant strain.

17. A method as claimed in claim 16, wherein the azole drug is selected from the group consisting of clotrimazole, flucanoazole, itracanoazole and micanazole.

18. A method as claimed in claim 13, wherein the fungus is simultaneously resistant to both polyene and azole classes of antibiotics.

19. A method a claimed in claim 7, wherein the subject is a human.

20. A method for the treatment of cancer in a subject comprising administering to the subject a pharmaceutically effective amount of methyl-β-orcinol carboxylate of formula I and a pharmaceutically acceptable carrier

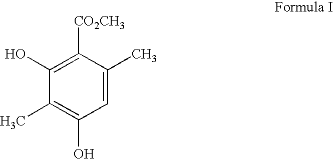

21. A method as claimed in claim 20, wherein the cancer comprises liver, colon, ovarian and mouth (oral) cancer.

22. A method as claimed in claim 20, wherein the subject is a human.

23. A method as claimed in claim 20, wherein the methyl-β-orcinol carboxylate of formula I is isolated form lichen Everniastrum cirrhatum.

24. A method as claimed in claim 20, wherein the concentration of methyl-β-orcinol carboxylate of formula I is in the range of 1-10 μg/ml.
